(19) United States
(12) Patent Application Publication
(10) Pub. No.: US 2018/0340151 A1
(43) Pub. Date: Nov. 29, 2018

(54) Method of making an artificial micro-gland that is anisotropic

(71) Applicants: Manuel Marquez, Midlothian, VA (US); Samantha M. Marquez, Midlothian, VA (US)

(72) Inventors: Manuel Marquez, Midlothian, VA (US); Samantha M. Marquez, Midlothian, VA (US)

(21) Appl. No.: 15/606,027

(22) Filed: May 26, 2017

(51) Int. Cl. C12N 5/071; C12N 13/00

(52) U.S. Cl. CPC C12N 5/0697; C12N 13/00

(57) ABSTRACT

A method is disclosed for making an artificial micro-gland having a continuous anisotropic membrane of two or more types of living cells. A first step includes forming a carrier fluid in a microchannel in a laminar flow of two distinct fluid flows. Another step includes introducing a template, which may itself be anisotropic, into the microchannel in a manner such that the template straddles the interface between the first fluid-flow and the second fluid-flow. In some embodiments two types of living cells within the template are separately attracted one of the fluid flows by the presence of an agent of taxis. In other embodiments, cells within one or the other of the fluid flows are attracted to agents within the template. Membranes form on the template and join together to form a complete cellular membrane around a reservoir.

### CROSS-REFERENCE TO RELATED APPLICATIONS

This application is a continuation of U.S. application Ser. No. 12/880,113 filed 12 Sep. 2010, which is a non-provisional of U.S. Provisional Application No. 61/260,824 filed 12 Nov. 2009; and also is a continuation-in-part of U.S. application Ser. No. 12/860,867 filed 21 Aug. 2010, which is a continuation in part of U.S. application No. 12/726,158 filed 17 Mar. 2010, which claims the benefit of U.S. Provisional Application No. 61/257,666 filed 3 Nov. 2009, and U.S. Provisional Application No. 61/165,989 filed 2 Apr. 2009, all of which are herein incorporated in their entirety by reference.

### TECHNICAL FIELD

In the field of bio-affecting and body-treating compositions, a method of making an artificial gland of micro-scale with a cellular membrane and bioreactor reservoir, wherein the artificial gland is useful for biological tissue and organ repair and replacement and stem cell engineering and biotechnology applications.

### BACKGROUND ART

The artificial micro-gland was first disclosed in applicant's patent applications, referenced above. The artificial micro-gland has a shell or membrane of living cells surrounding a core or reservoir. The term “living cells” is intended to broadly encompass biological units and cells as defined in parent patent application Ser. No. 12/726,158 for the artificial micro-gland. The more significant applications of the invention are currently expected to employ living cells comprising fungi, algae, bacteria and mammalian cells like fibroblasts and stem cells. The living cells may be one type or a multiple of types of cells.

The reservoir is a micro-volume bio-reactor that supports a biologically active environment. For example, it may host a medicinal component or biological activity creating helpful substances for promoting healing, vaccination, or food active ingredients. The micro-gland has potential application as a means for drug and/or cell delivery within human or other animal.

The present invention teaches a method of making the artificial micro-gland with two or more cell types, preferably each cell type in distinct patch or zone or spheroidal segment of the membrane, thus forming an artificial micro-gland that is anisotropic.

The method disclosed herein for making an artificial micro-gland employs taxis to stimulate living cell self-assembly into a continuous membrane, also referred to as a shell, surrounding a template. Once surrounded, the template becomes the reservoir of an artificial micro-gland.

Taxis is a form of tropism, which involves the stimulating the motility or migration of a cell or organism towards or away from a location by a physical condition or distinct difference between where the living cells are situated and an adjoining location. This may be referred to herein as an agent, an agent of taxis, or an agent of attraction.

Living cells are stimulated to form the shell usually at a liquid/liquid interface, a liquid/gel interface, or a liquid/gas interface. For simplicity herein, a cell or an organism may also be referred to as a living cell.

The prime examples of the method employ a laminar flow of two adjoining liquids within a microchannel or capillary. The method also applies to using more than two adjoining liquids.

Fluid flow at this scale is generally referred to as micro-fluidics, which involves the behavior of fluids at the micro-scale. Such behavior is significantly different than fluid flow at the macro-scale because it enables laminar flow of adjoining liquids due to the dominance of physical properties such as surface tension, energy dissipation, and fluidic resistance. In particular, the Reynolds number, which reflects the effect of momentum of a fluid compared to the effect of viscosity, reduces to a small value. This in turn means that fluids, when side-by-side, tend not to mix in the traditional macro sense. Rather, molecular transport between them tends to occur through diffusion. High specificity of chemical and physical properties (concentration, pH, temperature, shear force, etc.) can also be ensured resulting in more uniform reaction conditions and higher grade products in single and multi-step reactions.

The technique for creating laminar flow of two or more adjoining fluids is described in a paper published by SCIENCE magazine, vol. 285, on 2 Jul. 1999, and titled “Microfabrication Inside Capillaries Using Multiphase Laminar Flow Patterning,” which is herein incorporated by reference. Additionally, anisotropic modification of micrometer sized objects in a laminar flow of two liquids was reported in ADVANCED MATERIALS magazine, “Polyelectrolyte Micropatterning Using a Laminar-Flow Microfluidic Device,” 16, No. 5, 5 Mar. 2004,” which is herein incorporated by reference. While these techniques are known, no one has combined them with living cells motivated by taxis or suggested the creation of an artificial micro-gland.

The present invention takes the process of using of laminar flow within a microchannel a step further, using taxis, wherein living cells direct their movements to the surface of a droplet, bubble, gel, or combination thereof (herein called a template) straddling the intersection of the two adjoining liquids in the laminar flow. The term “fluid,” as used herein, can be a liquid or a gas. However, most embodiments will use liquid laminar flows and aqueous droplets of monodisperse living-cell suspensions in a solvent. The cells may be in the template or in either or both of the adjoining liquids in the laminar flow and are directed to form the membrane on the surface or boundary of the template with each of the two adjoining liquids. Since the template straddles the intersection of the two adjoining liquids, each liquid affects the zone of the spheroidal template differently and thus can form a membrane with different cells.

One or more agents influencing the movement of the living cells to the boundary with the template may be used. An agent may be another living cell that has a symbiotic relationship with the living cells present in either one or both of the adjoining liquids, or within the template.

The agent may be pH; temperature; responsiveness to light; electrical charge; responsiveness to a magnetic field; and, responsiveness to an electric field; or it may be a chemo-attractant, of which many are well known. Magnets, exposure laser light, exposure to heat variants, exposure to pH variants and exposure to an electric field are the most common.

Cells may move in response to the environment's magnetic characteristics is known as magnetotaxis, an instance of magnetoception. It is believed to aid these organisms in reaching regions of optimal oxygen concentration. The preference for organisms (bacteria, algae, yeast, etc) to move and concentrate in regions of specific temperatures is called: thermotropism. There are several examples of thermotrophic bacteria, thermotrophic yeast and other kind of cells.

Taxis may also take the form of chemotropism, movement or growth in response to chemicals; Gravitropism (or geotropism), movement or growth in response to gravity; Heliotropism, movement or growth in response to sunlight; Phototropism, movement or growth in response to lights or colors of light; Thermotropism, movement or growth in response to temperature; Thigmotropism, movement or growth in response to touch or contact; and, Host tropism or cell tropism, the host range of pathogens.

The agent affects metabolic activity of the living cells and so its presence in higher concentrations in the template selectively draws the cells to the interface of the template with the two adjoining liquids. Alternatively, a higher agent concentration outside the template in one or more fluid flows surrounding the template, draws living cells within the template to the interface with the fluid flow.

Iron, for example, is an essential metal for virtually all organisms. Iron acquisition is well characterized for various organisms, whereas intracellular iron distribution is poorly understood. In contrast to bacteria, plants, and animals, most fungi lack ferritin-mediated iron storage but possess an intracellular siderophore shown to be involved in iron storage. There are several examples of Cells and biological units that respond to magnetic fields. Even Fungi. The most popular biological system is the Magnetotactic bacteria.

Six alternative embodiments of using laminar flow in a microchannel of two adjoining liquids, forming a carrier fluid, with a template straddling the intersection of the two adjoining liquids are described. The embodiments vary the location of the living cells, the agent of attraction, and the template.

### SUMMARY OF INVENTION

A method is disclosed for making an artificial micro-gland having a continuous membrane of two or more types of living cells. An artificial micro-gland is formed by a continuous membrane defining an enclosed volume, the enclosed volume comprising a reservoir serving as a bioreactor. The artificial micro-gland produced by the method is anisotropic with respect to the cellular structure of the membrane. The membrane is composed of two or more distinct patches or zones of differing living cells.

The method employs a step of forming a carrier fluid in a microchannel in a laminar flow, the carrier fluid comprising two distinct fluid flows. The fluid flows adjoin each other so as to define an interface between the two flows.

In the several embodiments, there may be a distinct difference between the first fluid-flow and the second fluid-flow, which causes the first fluid-flow to attract first-type living cells and the second fluid-flow to attract a second-type living cells, or either or both of the two fluid flows may hold living cells.

The method includes a step of introducing a template, which may be a droplet, bubble, or gel of fluid, into the microchannel in a manner such that the template straddles the interface between the first fluid-flow and the second fluid-flow. When the template is a droplet of fluid, it may also contain living cells, such as the first-type living cells and the second-type living cells.

The method includes a step of retaining the template of fluid in the laminar flow until two partial membranes are formed by movement of at least one type of the living cells to the template zone adjoining the first fluid and at least another type of living cells adjoining the second fluid. When these two partial membranes join together to form a complete membrane around a reservoir comprising the template, then a stable artificial micro-gland is formed.

The method finally includes a step of removing the artificial micro-gland from the carrier fluid. Artificial micro-glands are living cellular spheroidal tissue, membrane assemblies or biofilms that define a core region, or reservoir, which may contain a solid, liquid, gas or nothing at all.

### Technical Problem

A method needs to be found to enable the fabrication of artificial micro-glands with distinct regions of differing living cells. This will add potentially diverse applications to healing and organ growth where adjacent symbiotic cells can provide favorable contributions to healing or cell growth. The anisotropic micro-gland can provide a means to control the application of chemicals, cells or biological units. This could take the form of highly specific drug delivery system, specific metabolite production by the shell, and/or preservation in the reservoir, which is also referred to as the core, and selective drug or cell delivery to a particular location within the body.

### Solution to Problem

Artificial micro-glands having an anisotropic membrane can be made to enable complex healing compositions. The method uses laminar flow of two adjoining liquids in a microchannel where a template is suspended between the two adjoining liquids in the laminar flow. Different cells within the template or within the fluid flows are then encouraged by taxis to form membranes in distinct regions of the interfaces of the template with the adjoining liquids. When these membranes join to surround the template, the artificial gland is made.

### Advantageous Effects of Invention

Complex patchy membranes of living cells in an artificial micro-gland can be fabricated in a single step and in situ. Ease of fabrication will provide a new tool in using artificial micro-glands: to address the complexities of the healing and cell growth processes; to design complex drug and cells delivery treatments; and to enable the creation of a multiplex of micro-bioreactors with multipletype cells using complex biofilm membranes.

The method can be used to create artificial micro-glands with either shape or composition anisotropies. When taxis is generated solely by permanent magnets, the process does not require additional energy, magnetic nanoparticles can be sequestered within specific regions leading to well oriented and easily manipulated artificial micro-glands. Electric fields from direct current or alternating current, and focused laser light at the micro scale can also be used. This method has direct implications on the development of molecular and synthetic biology toolsets for redesigning or de novo engineering of biological signaling networks.

### BRIEF DESCRIPTION OF DRAWINGS

The drawings illustrate preferred embodiments of the method of the invention. New reference numbers in FIG. 2 are given the 200 series numbers. Similarly, any new reference numbers in each succeeding drawing are given a corresponding series number beginning with the figure number.

FIG. 1 is a sectional view of a microchannel showing injection of two fluid flows and a droplet.

FIG. 2 is a sectional view of a droplet in a microchannel straddling the interface of two fluid flows and showing the position and movement of two types of living cells within the droplet.

FIG. 3 is a sectional view of a template (droplet, gel, or bubble) with one type of living cells in the first fluid-flow and another type in the second fluid-flow.

FIG. 4 is a sectional view of a droplet with one type of living cells in the first fluid-flow and two types of living cells within the droplet.

FIG. 5 is a sectional view of a droplet with one type of living cells in the first fluid-flow and another type in the second fluid-flow and one type of living cells within the droplet.

FIG. 6 is a sectional view of a droplet with one type of living cells in the first fluid-flow and another type in the second fluid-flow and two different living cells within the droplet.

FIG. 7 is a sectional view of an artificial micro-gland with two types of living cells in the membrane in well defined locations.

FIG. 8 is a sectional view of an artificial micro-gland with three types of living cells in the membrane in well defined locations.

FIG. 9 is a sectional view of an artificial micro-gland with four types of living cells in the membrane in well defined locations.

FIG. 10 is a sectional view of an artificial micro-gland with an anisotropic template.

FIG. 11 is a sectional view of an artificial micro-gland in a microchannel with an anisotropic template and two types of living cells in two fluid flows.

### DESCRIPTION OF EMBODIMENTS

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized and operational changes may be made, without departing from the scope of the present invention. For example, the steps in the method of the invention may be performed in any order that results making or using the artificial micro-gland.

The embodiments of the invention described herein are alternative methods of making an artificial micro-gland with an anisotropic membrane. An anisotropic membrane has patches or zones of differing living cells. A membrane may also be referred to as a biofilm. As in all of the methods disclosed herein, the artificial micro-gland comprises a continuous membrane of living cells surrounding and defining an enclosed volume. The enclosed volume comprises a reservoir serving as a bioreactor. This is the same artificial micro-gland as described in the parent application, U.S. application Ser. No. 12/726,158, filed 17 Mar. 2010.

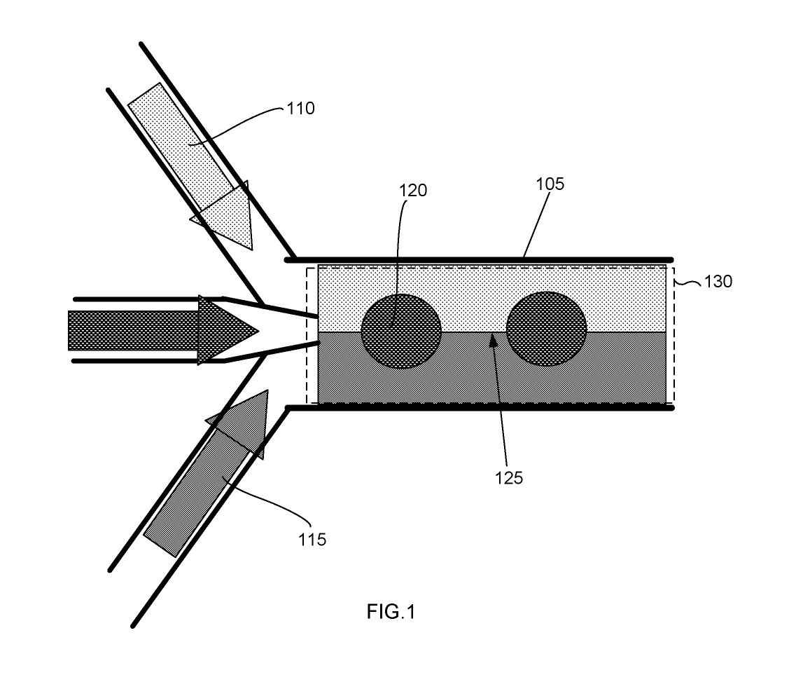

FIG. 1 illustrates a microchannel (105) device enabling the injection of a first fluid-flow (110), and a second fluid-flow (115) together comprising a laminar flow. A droplet (120), or more generically a template, is also injected to straddle the interface (125) between the two fluid flows. A template may be a droplet, bubble, gel, artificial micro-gland, or a combination of these; but a droplet, which is a liquid, is used when high living cell motility within the template is desired.

The embodiments disclosed herein differ in respect to where the living cells start out, what and where the agent of taxis is, and the resulting number of patches or zones of living cells in the membrane of the artificial micro-gland.

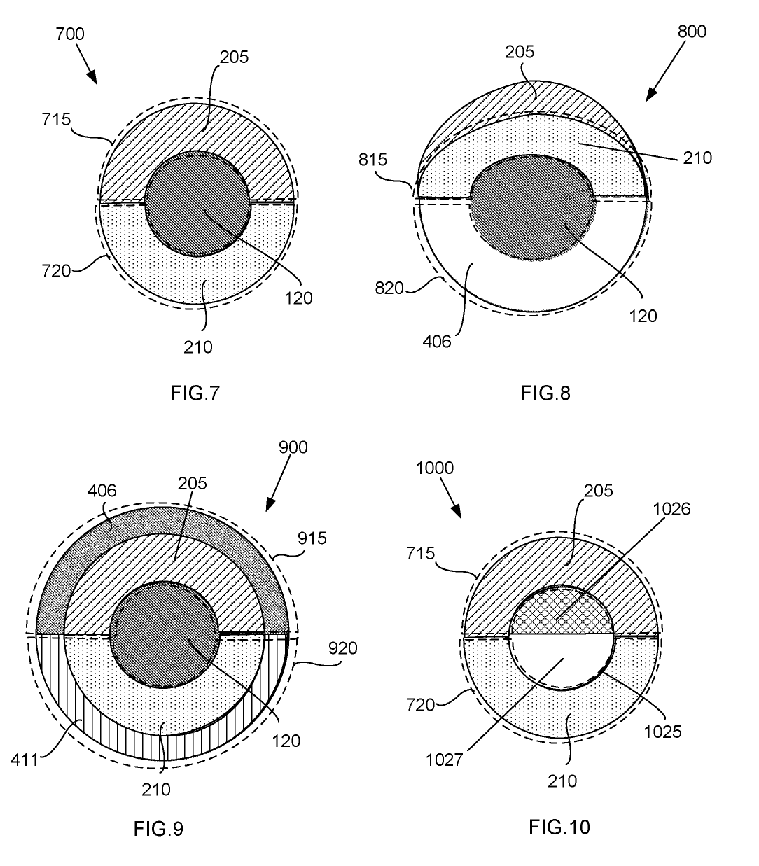

Preferred methods of making the artificial micro-gland include a continuous membrane of two or more types of living cells and the embodiments disclosed herein include a membrane with two, three and four different types of living cells. These are illustrated in FIGS. 7-10, which figuratively show a sectional view of these artificial micro-glands made according to the method of the invention: a two living-cell artificial micro-gland (700) is illustrated in FIG. 7; a three living-cell artificial micro-gland (800) is illustrated in FIG. 8; a four living-cell artificial micro-gland (900) is illustrated in FIG. 9; and, an anisotropic-template artificial micro-gland (1000) is illustrated in FIG. 10. Other variations with more living cell are possible if one starts with an artificial micro-gland as the template.

### First Embodiment

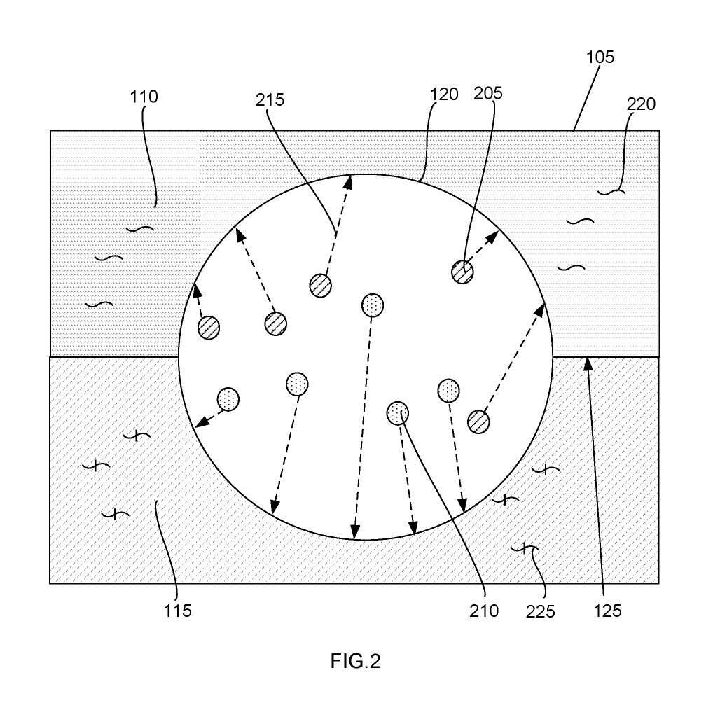

A first embodiment of the method of the invention is illustrated in FIG. 2. This first embodiment involves living cells in the template, which in this case is a droplet (120) of liquid. Two types of living cells are used. An agent selective for one of these cells type is present in each fluid flow. The living cells migrate to the junction with their respective fluid flows as attracted by the agent. A continuous membrane is formed comprising the two types of living cells.

This first embodiment includes a step of forming a carrier fluid (130), in a microchannel (105) in a laminar flow. As discussed above formation of such a laminar flow has been described in the literature.

The carrier fluid (130) is represented by the dashed enclosure with this reference number in FIG. 1. The laminar flow comprises two fluid flows: a first fluid-flow (110) and a second fluid-flow (115) adjoining the first fluid-flow (110) at an interface (125) of the two fluid flows.

There is a distinct difference between the first fluid-flow (110) and the second fluid-flow (115), which causes the first fluid-flow (110) to attract first-type living cells (205) and causing the second fluid-flow (115) to attract a second-type living cells (210).

The distinct difference may be a difference in a measurable property such as pH; temperature; responsiveness to light; electrical charge; responsiveness to a magnetic field; or, responsiveness to an electric field.

The distinct difference may also be due to the presence of an agent promoting taxis for a type of living cells, which presence may be in one or both of the first fluid-flow (110) and the second fluid-flow (115), or it may be simply a different agent in each of these two fluid flows. A first agent (220), figuratively represented by the squiggly line in FIG. 2, for the first-type living cells (205) may be in the first fluid-flow (110); and a second agent (225) for the second-type living cells (210) may be in the second fluid-flow (115). Such agents are well known and examples are oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

The living cells are preferably either eukaryotic cells or prokaryotic cells. Thus, the first-type living cells (205) and the second-type living cells (210) are selected from the group consisting of: eukaryotic cells; and, prokaryotic cells.

Another step in the first embodiment of the method of the invention is introducing a droplet (120) of fluid into the microchannel (105) in a manner such that the droplet (120) of fluid straddles the interface (125) between the first fluid-flow (110) and the second fluid-flow (115). Such straddling is enabled when the droplet (120) of fluid is poorly immiscible, or simply immiscible, in the first fluid-flow (110) and the second fluid-flow (115). The droplet (120) of fluid comprises first-type living cells (205) and second-type living cells (210).

Another step in the first embodiment of the method of the invention is retaining the droplet (120) of fluid in the carrier fluid (130) until a first-partial membrane (715) and a second partial membrane (720) are formed and joined together. The first-partial membrane (715) is indicated by the first-type living cells (205) within the dashed enclosure in FIG. 7 labeled with the reference number for the first-partial membrane (715). The second-partial membrane (720) is indicated by the second-type living cells (210) within the dashed enclosure in FIG. 7 labeled with the reference number for the second-partial membrane (720).

The first-partial membrane (715) formed by the first-type living cells (205) on a portion of surface of the droplet (120) of fluid in contact with the first fluid-flow (110).

The second-partial membrane (720) is formed by the second-type living cells (210) on a portion of surface of the droplet (120) of fluid in contact with the second fluid-flow (115). These partial membranes join together, that is, the second-partial membrane (720) joins with the first-partial membrane (715) to form a continuous membrane, and, thus forming an artificial micro-gland comprising a continuous membrane surrounding the droplet (120) of fluid.

Another step in the first embodiment of the method of the invention is removing the artificial micro-gland from the carrier fluid (130). Separation can be achieved by evaporation, pouring the carrier fluid (130) on a glass plate, or using a sieve. Such removal may include storing the artificial micro-gland in another liquid to enable preservation and monitoring vitality.

### Second Embodiment

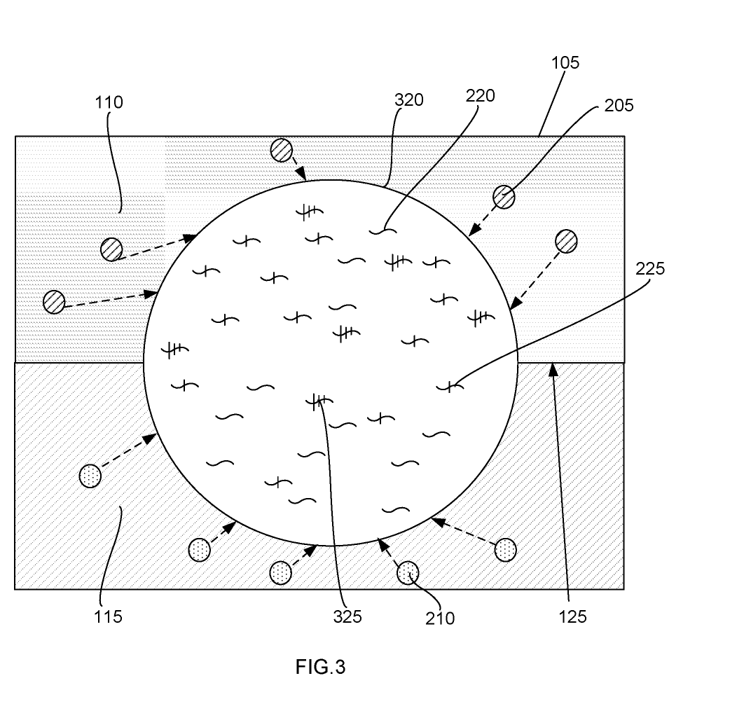

A second embodiment of the method of the invention is illustrated in FIG. 3. This second embodiment involves living cells in both fluid flows but not in the template, which may be a droplet, bubble, gel, or artificial micro-gland. Two types of living cells are used, one in each fluid flow. Each type of living cells migrate to the junction of their respective fluid flows with the template. Partial membranes formed by each cell type join together and form a continuous membrane comprising the two types of living cells, which surround the reservoir comprising the template.

The second embodiment includes a step of forming a carrier fluid (130) in a microchannel (105) in a laminar flow. This carrier fluid (130) comprises a first fluid-flow (110), which includes first-type living cells (205); and, a second fluid-flow (115) adjoining the first fluid-flow (110) at an interface (125), which includes second-type living cells (210).

The second embodiment includes a step of introducing a spheroidal template (320) into the microchannel (105) in a manner such that the spheroidal template (320) straddles the interface (125) between the first fluid-flow (110) and the second fluid-flow (115). Such straddling is enabled when the spheroidal template (320) is poorly immiscible, or simply immiscible, in the first fluid-flow (110) and the second fluid-flow (115). The spheroidal template (320) includes an agent promoting attraction (325) of the first-type living cells (205) and the second-type living cells (210). One agent may attract both types of living cells. However, it is more likely that a specific agent for each type of cell will be needed. When two agents are needed a first agent (220) and a second agent (225) are present in the spheroidal template (320). The spheroidal template (320) may be an ellipsoid or have a tubular shape, oval, or other three-dimensional shape.

The second embodiment includes a step of retaining the spheroidal template (320) in the carrier fluid (130) until a first-partial membrane (715) and a second partial membrane (720) are formed and joined together. The first-partial membrane (715) is formed by the first-type living cells (205) on a portion of surface of the spheroidal template (320) in contact with the first fluid-flow (110); and, a second-partial membrane (720) is formed by the second-type living cells (210) on a portion of surface of the spheroidal template (320) in contact with the second fluid-flow (115) such that the second-partial membrane (720) joins with the first-partial membrane (715) to form a continuous membrane, which surrounds the spheroidal template (320) and forms the two living-cell artificial micro-gland (700).

The second embodiment includes a step of removing the artificial micro-gland from the carrier fluid (130). Separation can be achieved by evaporation, pouring the carrier fluid (130) on a glass plate, or using a sieve. Such removal may include storing the artificial micro-gland in another liquid to enable preservation and monitoring vitality.

The living cells are preferably either eukaryotic cells or prokaryotic cells. Thus, the first-type living cells (205) and the second-type living cells (210) are selected from the group consisting of: eukaryotic cells; and, prokaryotic cells.

The agent promoting attraction (325) of the first-type living cells (205) and the second-type living cells (210) may be a physical parameter such as pH; temperature; responsiveness to light; electrical charge; responsiveness to a magnetic field; and, responsiveness to an electric field. The agent promoting attraction (325) may also be selected from the group of: oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

### Third Embodiment

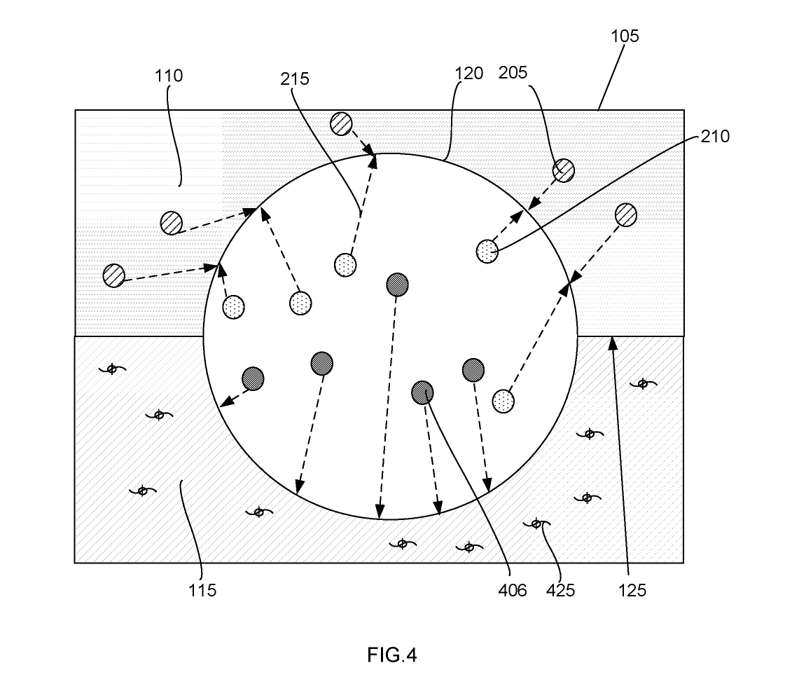

A third embodiment of the method of the invention is illustrated in FIG. 4 and the resulting three living-cell artificial micro-gland (800) is illustrated in FIG. 8. An artificial micro-gland with a continuous membrane of three or more types of living cells is possible with this method.

This third embodiment involves one type of living cells in one of the fluid flows, and two types of living cells in a droplet. Three types of living cells are used in all and what results is an artificial micro-gland with a membrane having two types of cells on one side and one type of living cells on the other. This is an artificial micro-gland with 3 patches or zones of differing living cells in its membrane. It may also be described as a membrane with one section formed by two-cell types and another section with one cell type.

One agent motivating taxis in this case is a symbiotic relationship, referred to as cooperation herein, between one type of the living cells within the droplet and the type of living cells within one of the fluid flows. This cooperation draws the two types of living cells together. Such cooperation pairs those two cells together. As before, this joining occurs at the interface of the droplet with the fluid flow containing the living cells. The other agent of taxis is within the fluid flow without the living cells, which encourages the other type of living cells to form a partial membrane with the interface with that fluid flow.

The third embodiment includes a step of forming a carrier fluid (130) in a microchannel (105) in a laminar flow. This carrier fluid (130) comprises a first fluid-flow (110), which includes first-type living cells (205); and, a second fluid-flow (115) adjoining the first fluid-flow (110) at an interface (125). The second fluid-flow (115) comprises an agent promoting taxis (425), preferably taxis of third-type living cells (406), which are the type of living cells within the droplet (120) introduced into the fluid flow in the another step.

The third embodiment includes a step of introducing a droplet (120) of fluid into the microchannel (105) in a manner such that the droplet (120) of fluid straddles the interface (125) between the first fluid-flow (110) and the second fluid-flow (115). Such straddling is enabled when the droplet (120) of fluid is poorly immiscible, or simply immiscible, in the first fluid-flow (110) and the second fluid-flow (115). The droplet (120) of fluid comprises a second-type living cells (210) and third-type living cells (406). The first-type living cells (205) and the second-type living cells (210) are attracted to each other, typically because of a symbiotic relationship.

The third embodiment includes a step of retaining the droplet (120) of fluid in the carrier fluid (130) until an upper-partial membrane (815) and a lower-partial membrane (820) are formed and joined together. These partial membranes are confined within the dashed enclosures designated with these reference numbers in FIG. 8. The use of the terms “upper” and “lower” have no position significance and are used only to differentiate these partial membranes from the first- and second-partial membranes discussed above.

The upper-partial membrane (815) is formed by joining together of first-type living cells (205) with second-type living cells (210) on a portion of surface of the droplet (120) of fluid in contact with the first fluid-flow (110).

The lower-partial membrane (820) is formed by third-type living cells on a portion of surface of the droplet (120) of fluid in contact with the second fluid-flow (115). The lower-partial membrane (820) joins with the upper-partial membrane (815) to form the three living-cell artificial micro-gland (800) in the carrier fluid (130).

The third embodiment includes a step of removing the three living-cell artificial micro-gland (800) from the carrier fluid (130). Separation can be achieved by evaporation, pouring the carrier fluid (130) on a glass plate, or using a sieve. Such removal may include storing the artificial micro-gland in another liquid to enable preservation and monitoring vitality.

The first-type living cells (205), the second-type living cells (210), and the third-type living cells (406) are preferably either eukaryotic cells or prokaryotic cells.

The agent promoting taxis (425) may be a physical parameter such as pH; temperature; responsiveness to light; electrical charge; responsiveness to a magnetic field; and, responsiveness to an electric field. The agent promoting taxis (425) may be selected from the group of consisting of: oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

### Fourth Embodiment

A fourth embodiment of the method of the invention is illustrated in FIG. 5 and the resulting three living-cell artificial micro-gland (800) is illustrated in FIG. 8. This is the same artificial micro-gland resulting from the third embodiment, but the method of getting to it is different.

This fourth embodiment involves living cells in each fluid flow and a single type of living cells in the droplet. Three types of living cells are used in all and what results is an artificial micro-gland with a membrane having two types of cells on one side and one type of living cells on the other. This is an artificial micro-gland with 3 patches or zones of differing living cells in its membrane.

One agent motivating taxis in this case is a symbiotic relationship, as in the third embodiment, between one type of the living cells within the droplet and the type of living cells within one of the fluid flows. This cooperation draws the two types of living cells together. Such cooperation joins those two cells together. As before, this joining occurs at the interface of the droplet with the fluid flow containing the living cells. The other agent of taxis is within the droplet, which encourages movement of the living cells within the droplet to form a partial membrane with the interface with that fluid flow.

The fourth embodiment includes a step of forming a carrier fluid (130) in a microchannel (105) in a laminar flow. This carrier fluid (130) comprises a first fluid-flow (110), which includes first-type living cells (205); and, a second fluid-flow (115) adjoining the first fluid-flow (110) at an interface (125). The second fluid-flow (115) comprises second-type living cells (210).

The fourth embodiment includes a step of introducing a droplet (120) of fluid into the microchannel (105) in a manner such that the droplet (120) of fluid straddles the interface (125) between the first fluid-flow (110) and the second fluid-flow (115). Such straddling is enabled when the droplet (120) of fluid is poorly immiscible, or simply immiscible, in the first fluid-flow (110) and the second fluid-flow (115). The droplet (120) of fluid includes third-type living cells (406). The droplet (120) of fluid further includes an agent promoting taxis (425) of the second-type living cells (210). The first-type living cells (205) and third-type living cells (406) are attracted to each other, typically by cooperation in a symbiotic relationship.

The fourth embodiment includes a step of retaining the droplet (120) of fluid in the carrier fluid (130) until an upper-partial membrane (815) is formed by joining together of first-type living cells (205) with second-type living cells (210) on a portion of surface of the droplet (120) of fluid in contact with the first fluid-flow (110).

This step further includes waiting until a lower-partial membrane (820) is formed by third-type living cells in the third type living cells (406). The lower-partial membrane (820) is formed on a portion of surface of the droplet (120) of fluid in contact with the second fluid-flow (115). The lower-partial membrane (820) joins with the upper-partial membrane (815) to form the three living-cell artificial micro-gland (800) in the carrier fluid (130). Here again, the use of the terms “upper” and “lower” have no position significance and are used only to differentiate these partial membranes from the first- and second-partial membranes discussed above.

The fourth embodiment includes a step of removing the artificial micro-gland from the carrier fluid (130). Separation can be achieved by evaporation, pouring the carrier fluid (130) on a glass plate, or using a sieve. Such removal may include storing the artificial micro-gland in another liquid to enable preservation and monitoring vitality.

The first-type living cells (205), the second-type living cells (210), and the third-type living cells (406) are preferably either eukaryotic cells or prokaryotic cells.

The agent promoting taxis (425) may be a physical parameter such as pH; temperature; responsiveness to light; electrical charge; responsiveness to a magnetic field; and, responsiveness to an electric field. The agent promoting taxis (425) may also be selected from the group consisting of: oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

### Fifth Embodiment

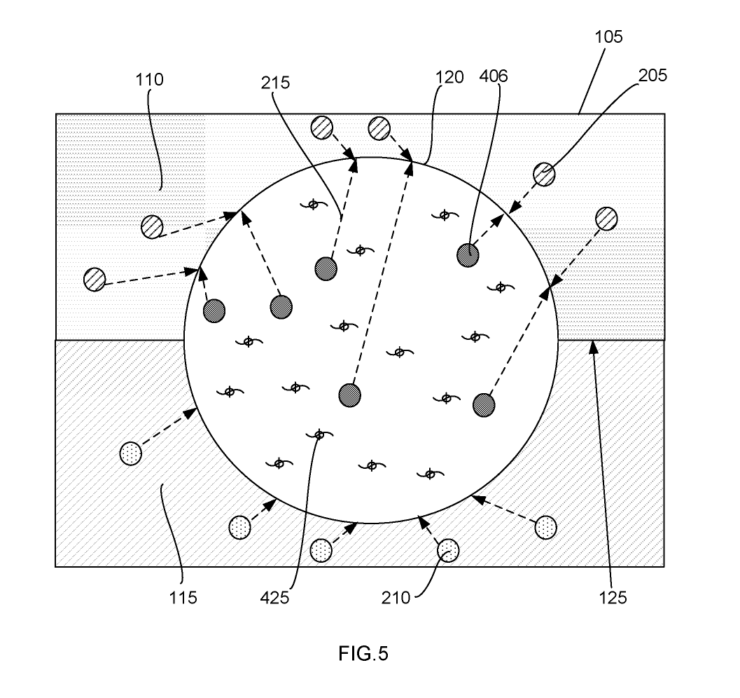

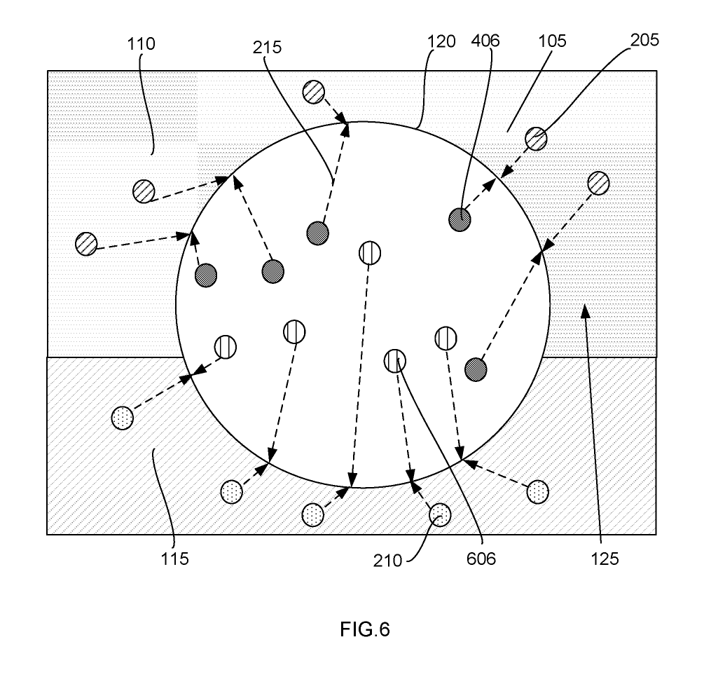

A fifth embodiment of the method of the invention is illustrated in FIG. 6 and the resulting four living-cell artificial micro-gland (900) is illustrated in FIG. 9. At least two continuous membranes are found in the fifth embodiment, which is created from two types of living cells, one in each fluid flow, and two types of living cells in a droplet. Four types of living cells are used in all and what results is an artificial micro-gland with two anisotropic membranes, namely an artificial micro-gland with 4 patches or zones of differing living cells in its two membranes. The agent motivating taxis in this case is a symbiotic relationship, referred to as cooperation herein, between one type of the living cells within the droplet and one type within one of the fluid flows. This cooperation draws the two types of living cells together. Such cooperation pairs those two cells together. Since there are two pairs cooperating, this forms two membranes. As before, this joining occurs at the interface of the droplet with the fluid flows.

The fifth embodiment includes a step of forming a carrier fluid (130) in a microchannel (105) in a laminar flow. This carrier fluid (130) comprises a first fluid-flow (110), which includes first-type living cells (205); and, a second fluid-flow (115) adjoining the first fluid-flow (110) at an interface (125), which includes second-type living cells (210).

The fifth embodiment includes a step of introducing a droplet (120) of fluid into the microchannel (105) in a manner such that the droplet (120) of fluid straddles the interface (125) between the first fluid-flow (110) and the second fluid-flow (115). Such straddling is enabled when the droplet (120) of fluid is poorly immiscible, or simply immiscible, in the first fluid-flow (110) and the second fluid-flow (115). The droplet (120) of fluid includes third-type living cells (406) and fourth-type living cells (606). The first-type living cells (205) and third-type living cells (406) are attracted to each other. The second-type living cells (210) and fourth-type living cells (606) are attracted to each other, typically because of a symbiotic relationship between them.

The fifth embodiment includes a step of retaining the droplet (120) of fluid in the carrier fluid (130) until a first-zone membrane (915) is formed by joining together of first-type living cells (205) with third-type living cells (406) on a portion of surface of the droplet (120) of fluid in contact with the first fluid-flow (110). The first-zone membrane (915) is indicated by the first-type living cells (205) and third-type living cells (406) approximately within dashed enclosure designated with the first-zone membrane (915) reference number.

This step further includes waiting until a second-zone membrane (920) is formed by joining together of second-type living cells (210) with fourth-type living cells (606) on a portion of surface of the droplet (120) of fluid in contact with the second fluid-flow (115), such that the second-zone membrane (920) joins with the first-zone membrane (915) to form a continuous membrane surrounding the droplet (120) of fluid. The second-zone membrane (920) is indicated by the second-type living cells (210) and the fourth-type living cells (606) approximately within dashed enclosure designated with the second-zone membrane (920) reference number.

This step further includes removing the continuous membrane surrounding the droplet (120) of fluid from the carrier fluid (130) to produce the four living-cell artificial micro-gland (900). Separation can be achieved by evaporation, pouring the carrier fluid (130) on a glass plate, or using a sieve. Such removal may include storing the artificial micro-gland in another liquid to enable preservation and monitoring vitality.

The first-type living cells (205), the second-type living cells (210), the third-type living cells (406), and the fourth-type living cells (406) are preferably either eukaryotic cells or prokaryotic cells.

### Sixth Embodiment

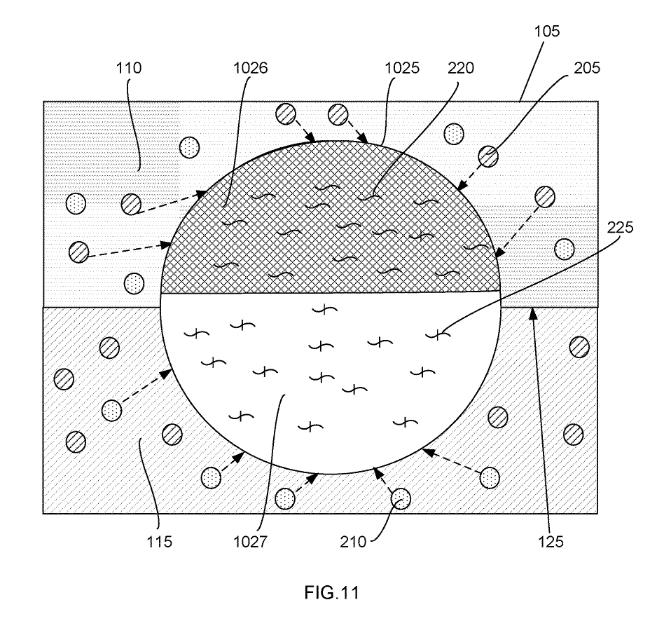

A sixth embodiment is illustrated in FIG. 10 and FIG. 11. The sixth embodiment utilizes an anisotropic template to create an anisotropic membrane with at least two different portions. For example, this anisotropic template may be a spheroidal droplet or gel comprising two contents that are asymmetrically distributed inside the droplet. The contents may be two chemicals situated separately from each other and within a single droplet. Formation of such anisotropic micro-scale droplets is discussed in: “Generation of Janus alginate hydrogel particles with magnetic anisotropy for cell encapsulation,” by L. B. Zhao, et al. and published by The Royal Society of Chemistry, 2009 Lab Chip, 2009, 9, 2981-2986, 2981; and further in “Multifunctional Superparamagnetic Janus Particles,” by Kai P. Yuet, et al. and published by the American Chemical Society, Langmuir 2010, 26(6), 4281-4287, published online 20 Oct. 2009, pubs. acs.org/Langmuir, DOI: 10.1021/1a903348s, both of which are herein incorporated by reference.

Each portion of the anisotropic template has its own agent and each flow has at least two types of cells, one attracted to each agent. As with each of the embodiments disclosed herein, the resulting artificial micro-gland comprises a continuous membrane of two or more types of living cells, the continuous membrane defining an enclosed volume, the enclosed volume comprising a reservoir serving as a bioreactor.

The sixth embodiment includes a step of forming a carrier fluid (130) in a microchannel (105) in a laminar flow. This carrier fluid (130) comprises a first fluid-flow (110), which includes first-type living cells (205) and second-type living cells (210); and, a second fluid-flow (115) adjoining the first fluid-flow (110) at an interface (125), which includes first-type living cells (205) and second-type living cells (210).

The sixth embodiment includes a step of introducing an anisotropic template (1025) into the microchannel (105) in a manner such that the anisotropic template (1025) straddles the interface (125) between the first fluid-flow (110) and the second fluid-flow (115).

The anisotropic template (1025) comprises a first fixed-volumetric portion (1026), which is represented by the cross-hatch in FIG. 10. The first fixed-volumetric portion (1026) comprises a first agent (220), which attracts first-type living cells (205). The first fixed-volumetric portion (1026) is essentially a region of the template having a specific chemical of physical property.

The anisotropic template (1025) further comprises a second fixed-volumetric portion (1027), which is shown in white in FIG. 10. The second fixed-volumetric portion (1027) comprises an agent promoting taxis (225) of second-type living cells (210).

The sixth embodiment includes a step of retaining the anisotropic template (1025) in the carrier fluid (130) until two conditions are satisfied: a first-partial membrane (715) is formed by first-type living cells (205) on a first surface of the anisotropic template (1025) comprising the first fixed-volumetric portion (1026); and, a second-partial membrane (720) is formed by second-type living cells (210) on a second surface of the anisotropic template (1025) comprising the second fixed-volumetric portion (1027) such that the second-partial membrane (720) joins with the first-partial membrane (715) to form an anisotropic-template artificial micro-gland (1000) in the carrier fluid (130), the anisotropic-template artificial micro-gland (1000) comprising a continuous membrane surrounding the anisotropic template (1025). In order to achieve this step, the two-fixed volumetric portions comprise distinct regions of the surface of the anisotropic template (1025) interfacing with the carrier fluid (130).

The sixth embodiment includes a step of removing the artificial micro-gland from the carrier fluid (130) to produce the anisotropic-template artificial micro-gland (1000). Separation can be achieved by evaporation, pouring the carrier fluid (130) on a glass plate, or using a sieve. Such removal may include storing the anisotropic-template artificial micro-gland (1000) in another liquid to enable preservation and monitoring vitality.

Other embodiments using an anisotropic template, a single type of living cells in each fluid flow, and a single agent of taxis in each portion of the anisotropic template are within the scope of the invention.

The terms “include” or “including” as used herein are not restrictive, but rather is open ended. These are intended to be equivalent to “comprise” or “comprising” and effectively mean “including, but not limited to.” The term “fluid,” as used herein may include a gas or a liquid. Typical fluids compatible with living cells are water and oil that may also include or contain nutrients or other additives compatible with the living cells.

The above-described embodiments including the drawings are examples of the invention and merely provide illustrations of the invention. Other embodiments will be obvious to those skilled in the art. Thus, the scope of the invention is determined by the appended claims and their legal equivalents rather than by the examples given.

### INDUSTRIAL APPLICABILITY

The invention has application to the biomedical and biotechnological industries.

1. A method of making a micro-capsule, the micro-capsule comprising a continuous membrane of two or more types of living cells, the continuous membrane defining an enclosed volume, the enclosed volume comprising a reservoir serving as a bioreactor, the method comprising the steps of:
forming a carrier fluid in a microchannel in a laminar flow, the carrier fluid comprising:
a first fluid-flow;
a second fluid-flow adjoining the first fluid-flow at an interface; and,
wherein, there is a distinct difference between the first fluid-flow and the second fluid-flow, said distinct difference causing the first fluid-flow to attract first-type living cells and causing the second fluid-flow to attract second-type living cells;
introducing a droplet of fluid into the microchannel in a manner such that the droplet of fluid straddles the interface between the first fluid-flow and the second fluid-flow, and, the droplet of fluid comprising first-type living cells and second-type living cells;
retaining the droplet of fluid in the carrier fluid until:
a first-partial membrane is formed by first-type living cells on a portion of surface of the droplet of fluid in contact with the first fluid-flow; and,
a second-partial membrane is formed by second-type living cells on a portion of surface of the droplet of fluid in contact with the second fluid-flow such that the second-partial membrane joins with the first-partial membrane to form the micro-capsule in the carrier fluid, the micro-capsule comprising a continuous membrane surrounding the droplet of fluid; and,
removing the micro-capsule from the carrier fluid.

2. The method of claim 1, wherein the distinct difference between the first fluid-flow and the second fluid-flow is a difference in a measurable property selected from the group consisting of: pH; temperature; responsiveness to light; electrical charge; responsiveness to a magnetic field; and, responsiveness to an electric field.

3. The method of claim 1, wherein the distinct difference between the first fluid-flow and the second fluid-flow is a difference created by the presence of an agent promoting taxis, the agent promoting taxis selected from the group consisting of: oxygen; carbon dioxide; nitrogen oxide; sugar; phosphates, nitrates, sulphates, and potassium salts; cyclic adenosine monophosphate (CAMP); inositon phospholipid (mPIP3); actin; histamine; serotonin (5HT); pialetet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

4. The method of claim 1, wherein the first-type living cells and the second-type living cells are selected from the group consisting of: eukaryotic cells; and, prokaryotic cells.

5. A method of making a micro-capsule, the micro-capsule comprising a continuous membrane of two or more types of living cells, the continuous membrane defining an enclosed volume, the enclosed volume comprising a reservoir serving as a bioreactor, the method comprising the steps of:
forming a carrier fluid in a microchannel in a laminar flow, the carrier fluid comprising:
a first fluid-flow, the first fluid-flow comprising first-type living cells;
a second fluid-flow adjoining the first fluid-flow at an interface, the second fluid-flow comprising second-type living cells;
introducing a spheroidal template into the microchannel in a manner such that the spheroidal template straddles the interface between the first fluid-flow and the second fluid-flow, and, the spheroidal template comprises an agent promoting attraction of first-type living cells and second-type living cells;
retaining the spheroidal template in the carrier fluid until:
a first-partial membrane is formed by first-type living cells on a portion of surface of the spheroidal template in contact with the first fluid-flow; and,
a second-partial membrane is formed by second-type living cells on a portion of surface of the spheroidal template in contact with the second fluid-flow such that the second-partial membrane joins with the first-partial membrane to form micro-capsule in the carrier fluid, the micro-capsule comprising a continuous membrane surrounding the spheroidal template; and,
removing the micro-capsule from the carrier fluid.

6. The method of claim 5, wherein the spheroidal template is selected from a group consisting of: a droplet of fluid; a gel; and, a bubble.

7. The method of claim 5, wherein the agent promoting attraction is selected from the group consisting of: pH; temperature; responsiveness to light; electrical charge; responsiveness to a magnetic field; and, responsiveness to an electric field.

8. The method of claim 5. wherein the agent promoting attraction is selected from the group consisting of: oxygen; carbon dioxide; nitrogen oxide; sugar;
phosphates, nitrates, sulphates, and potassium salts; cyclic adenosine monophosphate (cAMP); inositon phospholipid (mPIP3); actin; histamine; serotonin (5HT); platelet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, a chemo-attractant.

9. The method of claim 5, wherein the first-type living cells and the second-type living cells are selected from the group consisting of: eukaryotic cells; and, prokaryotic cells.

10. A method of making micro-capsule, the micro-capsule comprising a continuous membrane of three or more types of living cells, the continuous membrane defining an enclosed volume, the enclosed volume comprising a reservoir serving as a bioreactor, the method comprising the steps of:
forming a carrier fluid in a microchannel in a laminar flow, the carrier fluid comprising:
a first fluid-flow, the first fluid-flow comprising first-type living cells; and.
a second fluid-flow adjoining the first fluid-flow at an interface, the second fluid-flow comprising an agent promoting taxis;
introducing a droplet of fluid into the microchannel in a manner such that the droplet of fluid straddles the interface between the first fluid-flow and the second fluid-flow;
the droplet of fluid comprising second-type living cells and third-type living cells;
wherein first-type living cells and second-type living cells are attracted to each other;
wherein, the third-type living cells is affected by the agent promoting taxis;
retaining the droplet of fluid in the carrier fluid until:
an upper-partial membrane Is formed by joining together of first-type living cells with second-type living cells on a portion of surface of the droplet of fluid in contact with the first fluid-flow; and,
a lower-partial membrane is formed by third-type living cells on a portion of surface of the droplet of fluid in contact with the second fluid-flow, such that the lower-partial membrane joins with the upper-partial membrane to form the micro-capsule in the laminar flow, the micro-capsule comprising a continuous membrane surrounding the droplet of fluid; and,
removing the micro-capsule from the carrier fluid.

11. The method of claim 10, wherein the first-type living cells, the second-type living cells, and the third-type living cells are selected from the group consisting of: eukaryotic cells; and, prokaryotic cells.

12. A method of making a micro-capsule, the micro-capsule comprising a continuous membrane of three or more types of living cells, the continuous membrane defining an enclosed volume, the enclosed volume comprising a reservoir serving as a bioreactor, the method comprising the steps of:
forming a carrier fluid in a microchannel in a laminar flow, the carrier fluid comprising:
a first fluid-flow, the first fluid-flow comprising first-type living cells;
a second fluid-flow adjoining the first fluid-flow at an interface, the second fluid-flow comprising second-type living cells; and,
introducing a droplet of fluid into the microchannel in a manner such that the droplet of fluid straddles the interface between the first fluid-flow and the second fluid-flow;
the droplet of fluid comprising:
third-type living cells; and,
an agent promoting taxis of the second-type living cells;
wherein first-type living cells and third-type living cells are attracted to each other;
retaining the droplet of fluid in the carrier fluid until:
an upper-partial membrane is formed by joining together of first-type living cells with second-type living cells on a portion of surface of the droplet of fluid in contact with the first fluid-flow; and,
a lower-partial membrane is formed by third-type living cells on a portion of surface of the droplet of fluid in contact with the second fluid-flow, such that the lower-partial membrane joins with the upper-partial membrane to form the micro-capsule in the laminar flow, the micro-capsule comprising a continuous membrane surrounding the droplet of fluid; and,
removing the micro-capsule from the carrier fluid.

13. The method of claim 12, wherein the first-type living cells, the second-type living cells, and, the third-type living cells, are selected from the group consisting of: eukaiyotic cells; and, prokaryotic cells.

14. A method of making micro-capsule, the micro-capsule comprising at least two continuous membranes of four or more types of living cells, the continuous membranes defining an enclosed volume, the enclosed volume comprising a reservoir serving as a bioreactor, the method comprising the steps of:
forming a carrier fluid in a microchannel in a laminar flow, the carrier fluid comprising:
a first fluid-flow, the first fluid-flow comprising first-type living cells;
a second fluid-flow adjoining the first fluid-flow at an interface, the second fluid-flow comprising second-type living cells;
introducing a droplet of fluid into the microchannel in a manner such that the droplet of fluid straddles the interface between the first fluid-flow and the second fluid-flow;
the droplet of fluid comprising third-type living cells and fourth-type living cells;
wherein first-type living cells and third-type living cells are attracted to each other; and,
wherein second-type living cells and fourth-type living cells are attracted to each other;
retaining the droplet of fluid in the carrier fluid until:
a first-zone membrane is formed by joining together of first-type living cells with third-type living cells on a portion of surface of the droplet of fluid in contact with the first fluid-flow; and,
a second-zone membrane is formed by joining together of second-type living cells with fourth-type living cells on a portion of surface of the droplet of fluid in contact with the second fluid-flow, such that the second-zone membrane joins with the first-zone membrane to form a continuous membrane surrounding the droplet of fluid; and,
removing the continuous membrane surrounding the droplet of fluid from the carrier fluid to produce the micro-capsule.

15. The method of claim 14, wherein first-type living cells, second-type living cells, third-type living cells, and fourth-type living cells are selected from the group consisting of: eukaryotic cells; and, prokaryotic cells.

16. A method of making micro-capsule, the micro-capsule comprising a continuous membrane of two or more types of living cells, the continuous membrane defining an enclosed volume, the enclosed volume comprising a reservoir serving as a bioreactor, the method comprising the steps of:
forming a carrier fluid in a microchannel in a laminar flow, the carrier fluid comprising:
a first fluid-flow comprising first-type living cells and second-type living cells;
a second fluid-flow adjoining the first fluid-flow at an interface, the second fluid-flow comprising first-type living cells and second-type living cells;
introducing an anisotropic template into the microchannel in a manner such that the anisotropic template straddles the interface between the first fluid-flow and the second fluid-flow, the anisotropic template comprising:
a first fixed-volumetric portion comprising first agent, which attracts first-type living cells; and,
a second fixed-volumetric portion of the anisotropic template, the second fixed-volumetric portion comprising an agent promoting taxis of second-type living cells;
retaining the anisotropic template in the camel fluid until:
a first-partial membrane is formed by first-type living cells on a first surface of the anisotropic template comprising the first fixed-volumetric portion; and,
a second-partial membrane is formed by second-type living cells on a second surface of the anisotropic template comprising the second fixed-volumetric portion, such that the second-partial membrane joins with the first-partial membrane to form the micro-capsule in the carrier fluid, the artificial micro-gland comprising a continuous membrane surrounding the anisotropic template; and,
removing the artificial micro-gland lion) the carrier fluid.

17. The method of claim 16, wherein first-type living cells and second-type living cells are selected from the group consisting of: eukaryotic cells; and, prokaryotic cells.
